(19) United States
(12) Patent Application Publication
(10) Pub. No.: US 2023/0338179 A1
(43) Pub. Date: Oct. 26, 2023

(54) Device for Providing a Supporting Force for the Upper Extremities of a User, and an Exoskeleton

(71) Applicant: Hilti Aktiengesellschaft, Schaan (LI)

(72) Inventor: Simon KAESMANN, Muenchen (DE)

(21) Appl. No.: 17/762,616

(22) PCT Filed: Sep. 17, 2020
(86) PCT No.: PCT/EP2020/075965

§ 371 Date: Mar. 22, 2022

(30) Foreign Application Priority Data
Sep. 23, 2019 (EP) 19198859.1

(51) Int. Cl. A61F 5/01

(52) U.S. Cl. CPC A61F 5/013; A61F 2005/0197; A61F 2005/0132; A61F 2005/0179

(57) ABSTRACT

A device for providing a supporting force for a user's upper extremity. The device includes a supporting device, an arm strut, a rotary joint that connects the supporting device to the arm strut, a spring element that provides the supporting force where the supporting force is provided in a first mode and a second mode, a connecting element where the spring element is disposed between the supporting device and the connecting element, and a force transmission element that has a first end and a second end and is guidable about the connecting element. The first end is attached to a first suspension point on the supporting device in the first mode and the second end is attached to a second suspension point on the arm strut in the first mode. The first and second ends are attached to the second suspension point in the second mode.

### BACKGROUND AND SUMMARY OF THE INVENTION

The present invention relates to a device for providing a supporting force for the upper extremities of a user, and to an exoskeleton that comprises at least one such device. The main advantage of the invention is that, with the proposed device, a supporting force for the upper extremities of a user can be provided in two different force ranges. As a result, the proposed exoskeleton can be operated in two different operating states or modes and has a particularly broad performance spectrum compared with other exoskeletons. Preferably, the two operating states are allowed by the provision of a spring element that cooperates with a connecting element and a force transmission element. It is a departure from the prior art that two different force modes can be provided without it being necessary to use two spring elements, which can have in particular different spring constants. As a result, the structure of the device or of the exoskeleton can be significantly simplified compared with conventional devices that are known from the prior art, while having the same functionality, and so particularly robust devices or exoskeletons, suitable in particular for use on construction sites, can be provided with the invention, which can be used for example in the construction industry.

In modern working life, there are physically strenuous activities that can represent a health risk to the worker in question. These physically strenuous activities include in particular overhead work, in which the worker carries out activities above shoulder level. This work can be for example the assembly of complex industrial products such as motor vehicles. However, it can also include for example skilled manual activities, for example the processing of ceilings and high walls in the construction sector or on construction sites. Particularly for this reason, this overhead work represents a health risk for the worker in question because working and keeping the arms above the head are carried out counter to the force of gravity, wherein gravity, as is known, represents a force that is directed downward, i.e., in the direction of the center of the Earth. In this regard, the worker is particularly stressed when heavy loads have to be kept above the head or shoulders of the worker for a relatively long time, as is the case for example when carrying out work with electric tools in the construction industry.

In order to reduce the stresses for the worker, exoskeletons, as they are known, have been proposed in the prior art, with which the effect of the force of gravity can be at least partially alleviated. In conventional exoskeletons, this alleviation is provided by supporting forces that counteract gravity, i.e., in an “upward” spatial direction. As a result, for example the upper extremities of the worker are pushed upward or the upper extremities of the user of the exoskeleton are prevented from dropping below a previously defined height. For example, it is possible for the upper extremities to rest on an arm strut, which is locked for example in a plane at the height of the user's shoulders, wherein the locking plane is formed substantially orthogonally to an imaginary axis that extends substantially parallel to the spine of the user. Also known from the prior art are exoskeletons that can be operated for example in two operating states. The prior art proposes for example that supporting forces of different magnitudes can be provided in the different operating states. To this end, the prior art proposes combining a plurality of spring elements, which, depending on the set-up, have different resulting stiffnesses. These constructions with a plurality of spring elements have turned out to be highly susceptible to dirt or dust, however, and so they can be used only with difficulty for example on a construction site. Furthermore, the complicated structure caused by the two springs makes it even harder to use the known exoskeletons in the construction industry.

Therefore, it is an object of the present invention to overcome the above-described drawbacks of conventional supporting devices and to provide a device for providing a supporting force for the upper extremities of a user, and an exoskeleton, which have a simple structure and are particularly robust with respect to dust and soiling, such that they can be used in particular on construction sites, in skilled manual work or in the construction industry. Furthermore, the device to be provided and the exoskeleton to be provided are intended to be particularly lightweight so that they do not represent an additional weight load for the worker.

The object is achieved by the subject matter of the independent claim(s). Advantageous embodiments relating to the subject matter of the independent claim(s) can be found in the dependent claims. In particular, the object is achieved by a supporting device that can be operated in preferably two different operating states.

The object is achieved by a device for providing a supporting force for the upper extremities of a user, wherein the supporting force is provided in a first mode and in a second mode, and wherein the device comprises a supporting device and an arm strut, which are connected together via a rotary joint such that the arm strut is mounted so as to be rotatable about an axis of rotation with respect to the supporting device. The device is characterized in that it comprises a spring element for providing the supporting force, wherein the spring element is arranged between the supporting structure and a connecting element, wherein the device comprises a force transmission element that has a first end and a second end and is able to be guided about the connecting element, wherein the ends of the force transmission element are able to be attached to the supporting device at a first suspension point and to the arm strut at a second suspension point, wherein the first end of the force transmission element is attached to the first suspension point and a second end of the force transmission element is attached to the second suspension point in the first mode, and the first end and the second end of the force transmission element are attached to the second suspension point in the second mode.

It has been found that with the invention, the functionality whereby two different operating or supporting modes can be set can surprisingly also be provided by the use, proposed according to the invention, of a spring element that cooperates with a connecting element and a force transmission element. It is a departure from the prior art that two different force modes can be provided without it being necessary to use two or more spring elements. Those skilled in the art had previously assumed that such a functionality could be provided in particular with two springs with different stiffnesses. This usually results in a complicated structure of the exoskeleton with a large number of individual components, however. As a result of the invention, the structure of the proposed device or of the proposed exoskeleton can be significantly simplified compared with conventional devices that are known from the prior art, while having the same functionality, and so particularly robust devices or exoskeletons, suitable in particular for use on construction sites, can be provided with the invention, which are particularly suitable for use outdoors, for example on construction sites. In particular, it has been found that the devices and exoskeletons according to the invention are particularly less susceptible to the penetration of dust and moisture.

Use tests have furthermore shown that, as a result of the proposed construction using the spring element, the connecting element and the force transmission element, a particularly lightweight device or a particularly lightweight exoskeleton can be provided. This is achieved in particular by the provision of the one spring element, the weight of which is lower than that of the known two springs, which are frequently made of metal. Furthermore, it has been found that exoskeletons with the proposed device are particularly easy to handle. Both advantages increase the acceptance of exoskeletons by workers and users and in this way contribute toward reducing health risks for the worker and exoskeleton user and allowing him to have freedom from discomfort for a long time, in spite of physically strenuous work.

In the context of the invention, it is preferred for the force transmission element to be formed by a rope. The force transmission element has a first and a second end, wherein the two ends of the force transmission element can also preferably be referred to as rope ends when the force transmission element of the device is formed by a rope. The force transmission element can be produced from natural fibers or plastic, and is designed in particular to safely withstand tensile loads without tearing. In the context of the invention, it is preferred for the force transmission element to be formed in a substantially rigid manner, while the spring element is formed in a substantially elastic manner. Preferably, the elastic properties of the force transmission element and of the spring element differ significantly from one another in this respect. In the context of the invention, it is particularly preferred for the spring element to bring about the majority or larger part of the elastic deformation within the proposed device, while the elastic deformation that is caused by the force transmission element is negligibly small.

In the context of the invention, it is preferred for the connecting element to be formed by a deflection pulley. In the context of the invention, this is preferably also referred to as a pulley and serves to guide a rope or the like. A pulley preferably represents, in the context of the invention, a disk provided with a groove for deflecting a rope-like device. It can be used as a force converter and, in the context of the present invention, connects the spring element to the force transmission element. A corresponding exemplary embodiment of the invention, in which the connecting element is formed by a deflection pulley, is illustrated in FIG. 6. In this embodiment of the invention, it is preferred for the force transmission element to be guided in the groove of the pulley, wherein the pulley marks the lowest point with respect to the device that the force transmission element can take up. The term “lowest point” is not unclear to a person skilled in the art because a person skilled in the art knows that, in the context of the present invention, the force of gravity acting in the direction of the center of the Earth defines the “downward” spatial direction. Accordingly, objects that are at a smaller distance from the ground are arranged beneath other objects that are at a greater distance from the ground. Thus, in the context of the invention, the wording that the deflection pulley marks the lowest point with respect to the device that the force transmission element can take up means preferably that the major part of the force transmission element is arranged above the connecting element. Preferably, the connecting element is located substantially centrally with respect to the force transmission element or, in other words, divides the force transmission element into two halves. The two halves of the force transmission element can define a plane that lies substantially parallel to a back plane of the user, in particular when the user's arms are stretched out to the sides, as is illustrated for example in FIGS. 3 and 4. Preferably, the spring element is also located in this back plane of the device.

In the context of the invention, it may also be preferred for the connecting element to be formed by a connecting element that has for example a substantially cuboidal basic body. A corresponding example is illustrated in FIG. 6. The connecting element in this preferred configuration of the invention can be referred to preferably as a cuboidal connecting element. It has preferably two cutouts, wherein an upper cutout serves for fastening the spring element. For example, a loop that forms the upper end of the spring element can be suspended in the upper cutout of the cuboidal connecting element. A lower cutout of the connecting element can be used preferably for receiving the force transmission element. In particular, the lower cutout of the connecting element can provide a similar function to the groove of a deflection pulley, in which the force transmission element can be guided. In the context of the invention, the upper cutout is preferably characterized by an opening that faces upward, while the lower cutout of the connecting element is characterized by an opening that faces in the downward spatial direction. Preferably, the cuboidal connecting element, or the lower cutout thereof, also divides the force transmission element into two halves. In this embodiment of the invention, too, the halves can define a plane, wherein the plane defined by the halves of the force transmission element can be arranged substantially orthogonally to a back plane of the user and is determined by the position of the suspension points. In the context of the invention, it is preferred for the connecting element to be designed to split a force of the spring element into for example two force paths. In this case, a first force path acts preferably between the connecting element and the first suspension point and a second force path acts preferably between the connecting element and the second suspension point. If the connecting element splits the force of the spring element into two force paths, it is preferred in the context of the invention for the first force path and the second force path to have substantially the same magnitude. As a result, there is preferably a substantially identical force at each of the two suspension points, thereby ensuring that the supporting force is provided in a particularly even and back-protecting manner for the user or their arms.

In the context of the invention, the supporting device may also be referred to as a supporting structure. It can be configured preferably as a strut or as a bar and is located in particular between the hip belt of an exoskeleton, which comprises at least one proposed device, and an arm strut of the device. At its lower end, the supporting device can be joined to the hip belt preferably by way of a ball joint. In its upper region, the connection with the arm strut is established preferably via the rotary joint.

In a preferred embodiment of the invention, the device may comprise in particular a spring element for providing the supporting force, wherein the spring element is arranged between the supporting structure and a deflection pulley, wherein the device comprises a rope that is able to be guided about the pulley and the ends of which are able to be attached to the supporting device at a first suspension point and to the arm strut at a second suspension point, wherein a first rope end is attached to the first suspension point and a second rope end is attached to the second suspension point in the first mode, and the first rope end and the second rope end are attached to the second suspension point in the second mode. In this preferred embodiment of the invention, it may preferably be the case that a first rope end is attached to the first suspension point and a second rope end is attached to the second suspension point in the first mode, and the first rope end and the second rope end are attached to the second suspension point in the second mode.

In the context of the invention, it is preferred for one spring element of the device to be formed by an expander. In the context of the invention, an expander represents preferably a rope-like device that has elastic properties. For example, the spring element can comprise an elastic strap, a tensioning string, a rubber strap, a rubber rope or a rubber string, without being limited thereto. In an alternative embodiment, the spring element can also be formed by a spring, such as a helical spring. The spring element is located according to the invention between the supporting structure and a connecting element. In the context of the invention, it is preferred for the spring element to have a lower end and an upper end, wherein the lower end can be fastened to the supporting structure and the upper end of the spring element ends in the region of the connecting element. For example, the spring element can form a loop at its upper end, wherein the loop of the spring element can be guided around the connecting element from below. This configuration of the spring element is preferred in particular when the spring element is formed by a rope-like device such as an expander or an elastic rope. A corresponding example is illustrated in FIG. 6. It may also be preferred, in the context of the invention, for the spring element to be fastened to the connecting element by an additional fastening means, such as an eye, a hook or a bracket. This configuration of the spring element is preferred in particular when the spring element is formed by a spring, for example a helical spring. A corresponding example is illustrated in FIG. 5.

Provision is made according to the invention for the force transmission element to have two ends, wherein the ends can be attached to a first suspension point and a second suspension point. The first suspension point is located preferably in the region of the supporting structure. In the context of the invention, it is particularly preferred for the first suspension point to be located in an upper region of the supporting structure, wherein the upper region of the supporting structure lies in the vicinity of the rotary joint that connects the supporting structure to the arm strut. Provision is made according to the invention for the device to comprise a supporting device and an arm strut, which are connected together via the rotary joint such that the arm strut is mounted so as to be rotatable about an axis of rotation with respect to the supporting device. Preferably, the first suspension point is located beneath this rotary joint. A possible arrangement of the first suspension point is illustrated in FIG. 1. The second suspension point of the device is located preferably in the region of the arm strut. In the context of the invention, it is particularly preferred for the second suspension point to be arranged at an inner end of the arm strut. Preferably, the rotary joint divides the arm strut into a longer outer region and a shorter inner region.

The outer region of the arm strut is located preferably in an outer region of the torso or the back of the user, wherein this outer region is in particular away from the spine of the user. The outer region of the arm strut can preferably also be referred to as the distal region of the arm strut. Preferably, the distal region of the arm strut has a greater length than the inner region of the arm strut, which, in the context of the invention, can preferably also be referred to as the proximal region of the arm strut. The inner or proximal region of the arm strut is preferably close to the spine of the user, wherein the second suspension point, in a particularly preferred embodiment of the invention, can be located at the inner end of the arm strut. In other words, the second suspension point is located in the immediate vicinity of the spine of the user.

A particular advantage of the proposed invention is that, with the present invention, a preferably passive exoskeleton can be provided, which can be operated or used in two operating modes. These operating modes are referred to preferably as the first and second mode in the context of the invention. In the context of the invention, it is preferred for the first mode to correspond to a first force range and the second mode to correspond to a second force range, wherein the user of the exoskeleton experiences supporting forces of different magnitudes, for example when carrying out overhead work, in the different modes. In the context of the invention, it is particularly preferred for supporting forces of different magnitudes to be able to be provided to the user with the present invention, depending on the set mode, when carrying out overhead work. The provision of the supporting forces of different magnitudes is allowed in particular by the different suspension of the force transmission element ends at the first and/or the second suspension point. Provision is made according to the invention for a first end of the force transmission element to be attached to the first suspension point and a second end of the force transmission element to be attached to the second suspension point in the first mode. This arrangement of the force transmission element is illustrated for example in FIGS. 1 and 3. In this first mode, a lower supporting force is provided to the user, on account of the specific suspension of the force transmission element, than in the second mode. In the second mode or in the second operating state of the device or of the exoskeleton, the first end and the second end of the force transmission element are attached to the second suspension point. The specific arrangement of the force transmission element according to the second operating or force mode is illustrated for example in FIGS. 2 and 4 and advantageously allows a particularly great supporting force for the user.

In the context of the invention, it is preferred for the first mode to correspond to a first force range and the second mode to correspond to a second force range. For example, the worker can be provided with a lower supporting force for the upper extremities in the first mode than in the second mode. The suspension in the first mode can preferably also be described as the force transmission element being suspended at the first and at the second suspension point. In the second mode, the two ends of the force transmission element are fastened preferably at the same suspension point, in particular the second suspension point. As a result, advantageously a greater supporting force can be generated than in the first mode or operating state of the device.

The device comprises a supporting device and an arm strut, wherein the supporting device is preferably arranged substantially parallel to an imaginary axis, wherein the axis extends substantially parallel to the spine of the user. The term “substantially” does not result in a lack of clarity for a person skilled in the art because a person skilled in the art knows that, when carrying the device or the exoskeleton, a deviation from exact mathematical parallelism can occur, this still being intended to be covered by the wording “substantially parallel”, however, in the context of the invention. In the context of the present invention, it is preferred for the supporting structure to have a lower end and an upper end, wherein the upper end leads to the rotary joint. The lower end can preferably end in a torso region of the user of the device. The proposed device can be used preferably in an exoskeleton, wherein the exoskeleton can preferably comprise a hip belt, to which the supporting structure is joined. The junction of the supporting structure with the hip belt or the connection between the hip belt and supporting structure can comprise for example a joint, which can be for example in the form of a ball joint. The use of ball joints at this point of an exoskeleton has been found to be advantageous because the user is provided with mobility or freedom of movement in as many directions as possible, i.e., preferably with a large number of degrees of freedom.

Provision is made according to the invention for the device to comprise a supporting device and an arm strut, which are connected together via the rotary joint such that the arm strut is mounted so as to be rotatable about an axis of rotation with respect to the supporting device. In the context of the invention, it is preferred for the axis of rotation of the rotary joint to be arranged substantially orthogonally to the imaginary axis, which extends substantially parallel to the spine of the user. The axis of rotation projects preferably out of a back plane of the user, wherein the back plane is defined substantially by the back surface of the user. Preferably, the axis of rotation of the rotary joint is formed substantially orthogonally to the back plane of the user, or the axis of rotation is substantially perpendicular to the back plane of the user. As a result, the arm strut is formed advantageously in a rotatable manner with respect to the supporting structure, wherein the rotary movement of the arm strut can take place in particular in the back plane.

In the context of the invention, it is preferred for the spring element to be designed to generate a torque about the axis of rotation. In the context of the invention, it is preferred for the spring element to provide a supporting force for the user, wherein the supporting force acts preferably on the arm strut. As a result, a torque is advantageously generated about the axis of rotation. Preferably, the arm strut is mounted so as to be rotatable about the axis of rotation with respect to the supporting structure.

In the context of the invention, it is preferred for the arm strut to have a receiving device for an upper extremity, wherein the receiving device can preferably be in the form of an armrest. Preferably, the receiving device is arranged in the outer region of the arm strut, i.e., it is at a large distance from the spine of the user. As a result of this large distance of the receiving device from the spine, the supporting action of the proposed device is perceived as particularly load-relieving by the user and the carrying of the device is perceived as particularly comfortable. An example of an arrangement of the armrest is illustrated for example in FIGS. 1 to 4. The receiving device is designed in particular to receive the upper extremities of the user of the device or of the exoskeleton. The upper extremities of the user are preferably the arms of the user. The device and the proposed exoskeleton are preferably carried by a user on their body such that in particular their upper arms are received by the receiving device. In the context of the invention, it is particularly preferred for a lower region of the upper arms to rest in the armrest of the arm strut. The lower region of the upper arm is preferably that region of the upper arm that starts at the elbow or is arranged directly above the elbow.

In a second aspect, the invention relates to an exoskeleton for supporting the upper extremities of a user. The exoskeleton is characterized in that it comprises one or more of the proposed devices. Schematic depictions of exemplary embodiments of the exoskeleton are illustrated in FIGS. 3 and 4. The definitions, technical effects and advantages that have been described for the device apply analogously to the exoskeleton, and vice versa. Preferably, the proposed exoskeleton is a passive exoskeleton, which comprises in particular no active components such as motors or drive means. In the context of the invention, it is preferred for the devices, in the context of the proposed exoskeleton, to be arranged substantially symmetrically to a sagittal plane, in particular when the exoskeleton comprises two devices. This preferably means, in the context of the invention, that the devices are arranged in an axially symmetric manner about an axis that extends substantially parallel to the spine. The devices can be fastened to one another and to a user by various fastening means such as belts, harnesses, straps, clasps or the like. For example, the exoskeleton can comprise a hip belt, at which the lower ends of the supporting devices are arranged. In the context of the invention, it is preferred for the supporting devices to be able to be fastened to the hip belt by means of ball joints. The regions of the hip belt to which the supporting devices are joined can be referred to preferably as connecting regions. Preferably, the connection between the hip belt and the spring elements of the proposed device also occurs in these connecting regions. These are preferably fastened to a lower region of the supporting device, wherein this lower region of the supporting device can coincide at least partially with the connecting region between the hip belt and the supporting devices. Preferably, the connecting region comprises the ball joints, with which the supporting devices can be fastened to the hip belt. The use of ball joints allows a particularly high level of mobility for the user, such that they are restricted as little as possible in terms of freedom of movement by the device or the exoskeleton. The exoskeleton can also comprise for example a means for fastening the exoskeleton or the device(s) to the upper body. This can be for example a chest belt, which can be arranged for example at the height of the connecting elements or slightly below the connecting elements. One possible embodiment of the exoskeleton with a chest strap is illustrated for example in FIGS. 3 and 4.

Preferably, as a result of the connection of two devices, an exoskeleton is obtained with which a supporting force for both upper arms of the user can be provided. However, exoskeletons that have for example only one device are also conceivable. This may be useful in some applications when, during overhead work, only one arm is used to carry out the work or when the user can use only one arm because the second arm is, for example, injured or missing as a result of an injury or an accident.

In one exemplary embodiment, which is described in the following text, the invention relates in particular to a passive exoskeleton for the upper extremities, wherein the exoskeleton provides a supporting force in order to at least partially compensate for gravitational forces. The device or the exoskeleton consists of an arm strut, which is connected to the upper arm of the user via an armrest, and a supporting structure, which is connected to the torso and/or upper body of the user. The arm strut is mounted so as to be rotatable about an axis of rotation with respect to the supporting structure. Between the arm strut and the supporting structure, a spring element provides a supporting force that acts on the arm strut and thus generates a torque about the axis of rotation. The spring element is connected to the supporting structure at one end and to a deflection pulley, which acts as a connecting element, at another end. A rope is wrapped, as a force transmission element, about the pulley, wherein the ends of the rope can be connected to a first and a second suspension point. The invention has advantageously two different operating states, which have different supporting forces. In operating state 1 (first mode), one rope end is connected to the arm strut (second suspension point) and the other rope end is connected to the supporting structure (first suspension point). In operating state 2 (second mode), both rope ends are connected to the arm strut at the second suspension point, with the result that a greater supporting force is allowed.

Further advantages of the invention will become apparent from the following description of the figures. The figures, the description and the claims contain numerous features in combination. A person skilled in the art will expediently also consider the features individually and combine them to form useful further combinations.

In the figures, identical and similar components are denoted by the same reference signs.

### BRIEF DESCRIPTION OF THE DRAWINGS

FIG. 1 shows a schematic illustration of a preferred configuration of the device in the first mode;

FIG. 2 shows a schematic illustration of a preferred configuration of the device in the second mode;

FIG. 3 shows an illustration of a preferred configuration of the exoskeleton in the first mode;

FIG. 4 shows an illustration of a preferred configuration of the exoskeleton in the second mode;

FIG. 5 shows different preferred configurations of the connecting element and of the spring element; and

FIG. 6 shows different preferred configurations of the connecting element and of the spring element.

### DETAILED DESCRIPTION OF THE DRAWINGS

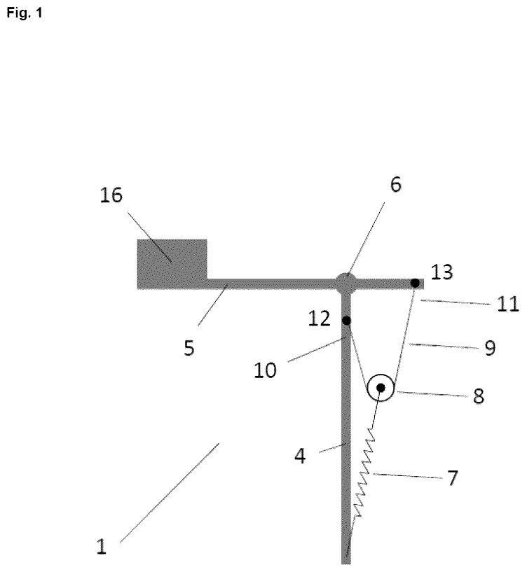

FIG. 1 shows a schematic illustration of a preferred configuration of the device (1) in the first mode, in which preferably a lower supporting force for the user (3) is provided than in the second mode. Illustrated in FIG. 1 is the arm strut (5), which is connected via a rotary joint (6) to a supporting device (4) or supporting structure (4). The arm strut (5) is mounted in a rotatable manner with respect to the supporting device (4), specifically about an axis of rotation that passes through the rotary joint (6). The supporting device (4) extends preferably substantially parallel to the spine of the user (3). From the supporting device (4) there extends a spring element (7), the upper end of which is fastened to a connecting means (8). In other words, the spring element (7) is arranged between the supporting structure (4) and the connecting means (8). The connecting means (8) can be in the form for example of a deflection pulley. It is preferably designed so that a force transmission element (9) can be guided around the connecting means (8). To this end, the connecting means (8) preferably has a groove or a slot. The force transmission means (9) can be in the form for example of a rope. It has two ends (10, 11), wherein the two ends (10, 11) of the force transmission element (9) can be attached to two suspension points (12, 13). The first suspension point (12) is arranged preferably on the supporting device (4), while the second suspension point (13) is arranged at an innermost end of the arm strut (5). The rotary joint (6) divides the arm strut (5) preferably into an outer region (14) and an inner region (15). In the context of the invention, it is preferred for the outer region (14) to have a greater length than the inner region (15) of the device (1). Preferably, the second suspension point (13) is located at the outermost end of the inner region (15) of the arm strut (5), that is to say particularly close to the spine of the user (3). In the exemplary embodiment illustrated in FIG. 1, the device (1) is in the first mode, in which the first end (10) of the force transmission means (9) is fastened to the first suspension point (12), while the second end (11) of the force transmission means (9) is fastened to the second suspension point (13). As a result of this arrangement, a low supporting force is transmitted to the upper extremities (2) of the user (3). The arm strut (5) of the device (1) may comprise a receiving device (16), which can be in the form for example of an armrest. The user (3) can rest for example their upper arm (2) there, in order to be able to work comfortably overhead.

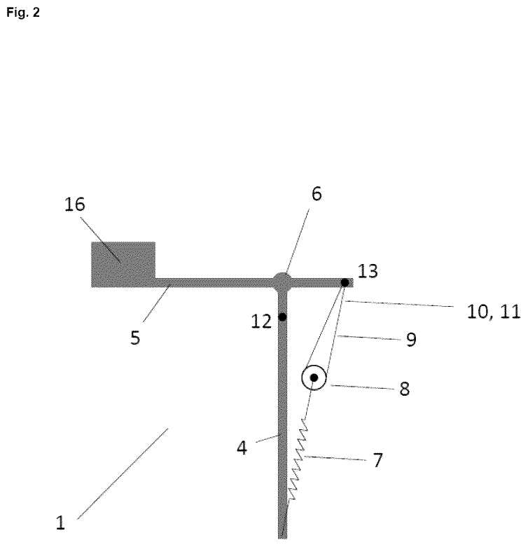

FIG. 2 shows a schematic illustration of a preferred configuration of the device (1) in the second mode. Unlike in FIG. 1, in the second mode or operating state of the device (1), both ends (10, 11) of the force transmission element (9) are fastened to the second suspension point (13) of the device, wherein the second suspension point (13) is preferably a constituent part of the arm strut (5). As a result, a greater supporting force can advantageously be provided for the user than in the first mode of the device (1).

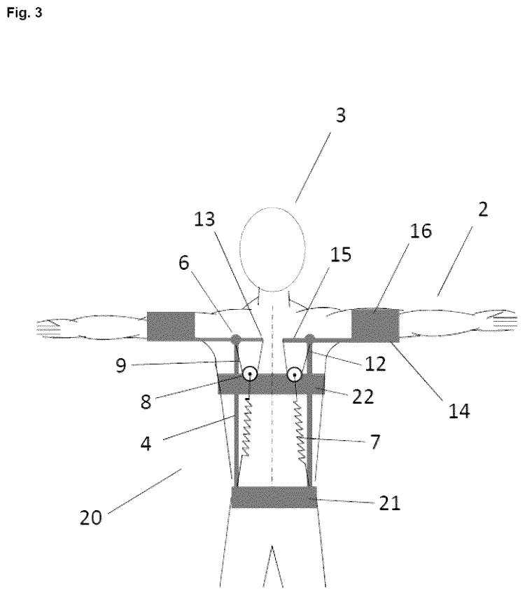

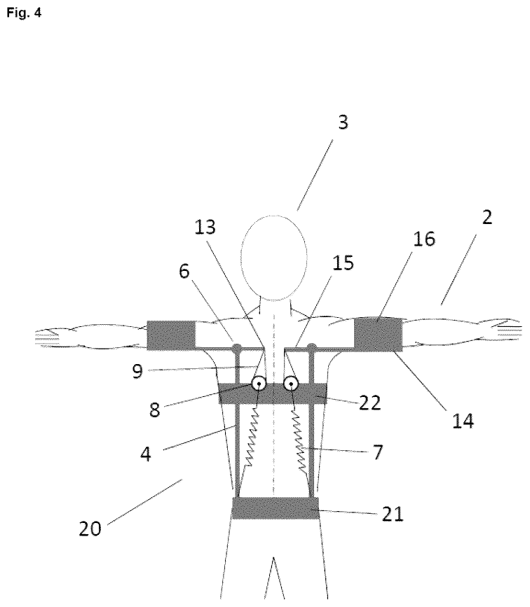

FIG. 3 shows an illustration of a preferred configuration of the exoskeleton (20) in the first mode. The exoskeleton (20) illustrated in FIG. 3 comprises two proposed devices (1), and also a hip belt (21) and a chest strap (22) or chest belt. The hip belt (21) can be placed around the hips by a user (3) and fastened and closed in a known way. As a result, at least a part of the weight of the exoskeleton (20) is supported on the hips of the user (3). The chest belt (22) is arranged preferably at the height of the chest of the user (3), wherein the connecting means (8) of the devices (1) are preferably also arranged at this height. In the state of the exoskeleton (20) illustrated in FIG. 3, the supporting devices (4) extend substantially perpendicularly upward from the hip belt (21), wherein the supporting devices (4) can be fastened to the hip belt (21) in particular by using ball joints (not illustrated). Preferably, the supporting structures (4) in the configuration of the device (1) illustrated in FIG. 3 extend substantially parallel to the imaginary axis that extends substantially parallel to the spine of the user. The imaginary axis is indicated in FIGS. 3 and 4 by the central dashed line. Between each connecting means (8) and the lower region of the supporting devices (4) there is arranged in each case a spring element (7), which may comprise or be formed by, for example, an elastic strap or a spring, such as a spiral spring or tension spring. Coming from above, the force transmission element (9) is guided around the connecting element (8). In the first mode of the exoskeleton (20) illustrated in FIG. 3, the first end (10) of the force transmission means (9) is fastened to the first suspension point (12), while the second end (11) of the force transmission means (9) is fastened to the second suspension point (13). Preferably, the first suspension point (12) is a constituent part of the supporting device (4), while the second suspension point (13) is arranged on the arm strut (5) in the immediate vicinity of the spine of the user (3).

FIG. 4 shows an illustration of a preferred configuration of the exoskeleton (20) in the second mode. In the second mode of the exoskeleton (20) illustrated in FIG. 4, both ends (10, 11) of the force transmission element (9) are fastened to the second suspension point (13) of the device, with the result that, advantageously, a greater supporting force for the user can be provided than in the first mode of the device (1).

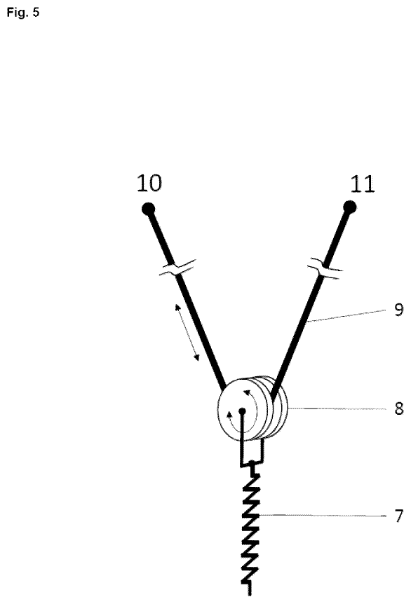

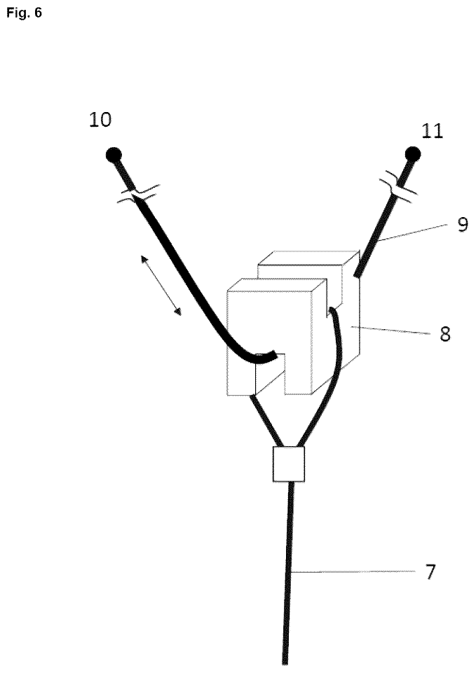

FIGS. 5 and 6 show different preferred configurations of the connecting element (8) and of the spring element (7). In particular, FIGS. 5 and 6 show the force transmission element (9), and also the first end (10) and the second end (11) thereof. In the exemplary embodiment of the invention shown in FIG. 5, the spring element (7) is in the form of a helical spring, while the connecting element (8) is formed by a deflection pulley. The force transmission element (9) is formed by a rope. In the exemplary embodiment of the invention shown in FIG. 6, the spring element (7) is formed by an expander, while the connecting element (8) comprises a cuboidal basic body. The force transmission element (9) is formed by a rope. It goes without saying that the different embodiments of the invention that are illustrated in FIGS. 5 and 6 can be combined with one another as desired.

### LIST OF REFERENCE CHARACTERS

1 Device
2 Upper extremity
3 User
4 Supporting device
5 Arm strut
6 Rotary joint
7 Spring element
8 Connecting element
9 Force transmission element
10 First end of the force transmission element
11 Second end of the force transmission element
12 First suspension point
13 Second suspension point
14 Outer region of the arm strut
15 Inner region of the arm strut
16 Receiving device
20 Exoskeleton
21 Hip belt
22 Chest belt

1.-14. (canceled)

15. A device for providing a supporting force for an upper extremity of a user, comprising:
a supporting device;
an arm strut;
a rotary joint, wherein the supporting device is connected to the arm strut via the rotary joint such that the arm strut is rotatable about an axis of rotation with respect to the supporting device;
a spring element, wherein the spring element provides the supporting force and wherein the supporting force is provided in a first mode and in a second mode;
a connecting element, wherein the spring element is disposed between the supporting device and the connecting element; and
a force transmission element that has a first end and a second end and is guidable about the connecting element, wherein the first end is attached to a first suspension point on the supporting device in the first mode and the second end is attached to a second suspension point on the arm strut in the first mode, and wherein the first end and the second end are attached to the second suspension point in the second mode.

16. The device as claimed in claim 15, wherein the supporting device has a lower end and an upper end and wherein the upper end leads to the rotary joint.

17. The device as claimed in claim 15, wherein a torque is generatable about the axis of rotation by the spring element.

18. The device as claimed in claim 15, wherein the rotary joint divides the arm strut into an outer region and an inner region and wherein the outer region is longer than the inner region.

19. The device as claimed in claim 15, wherein the arm strut has a receiving device for the upper extremity of the user.

20. The device as claimed in claim 19, wherein the receiving device is disposed in an outer region of the arm strut.

21. The device as claimed in claim 15, wherein the second suspension point is disposed at an inner end of the arm strut.

22. The device as claimed in claim 15, wherein the axis of rotation is disposed orthogonally to an imaginary axis and wherein the imaginary axis extends parallel to a spine of the user when the device is worn by the user.

23. The device as claimed in claim 15, wherein a force of the spring element is splittable into a first force path and a second force path by the connecting element and wherein the first force path acts between the connecting element and the first suspension point and the second force path acts between the connecting element and the second suspension point.

24. An exoskeleton, comprising:
a first device as claimed in claim 15; and
a second device as claimed in claim 15.

25. The exoskeleton as claimed in claim 24, wherein the first and the second devices are disposed symmetrically to a sagittal plane of a user when worn by the user.

26. The exoskeleton as claimed in claim 24, further comprising a hip belt, wherein respective lower ends of the respective supporting devices of the first and the second devices are disposed at the hip belt.

27. The exoskeleton as claimed in claim 26, wherein the respective supporting devices are fastenable to the hip belt by respective ball joints.

28. The exoskeleton as claimed in claim 24, further comprising a chest belt and wherein the chest belt is fastenable to a user of the exoskeleton.
